(19) United States
(12) Patent Application Publication
(10) Pub. No.: US 2026/0334868 A1
(43) Pub. Date: Oct. 29, 2026

(54) SYSTEMS AND METHODS FOR COLLABORATIVE SLEEP THERAPY USAGE

(71) Applicant: RESMED DIGITAL HEALTH INC., San Diego, CA (US)

(72) Inventors: Michael James DENT, Bella Vista, New South Wales (AU); Jaiden James CHOY, Bella Vista, New South Wales (AU); Andrew CHAN, Bella Vista, New South Wales (AU); Jamie Graeme WEHBEH, Bella Vista, New South Wales (AU)

(21) Appl. No.: 19/103,744

(22) PCT Filed: Aug. 17, 2023
(86) PCT No.: PCT/US2023/030459

§ 371 Date: Feb. 13, 2025

Related U.S. Application Data
(60) Provisional application No. 63/371,973, filed on Aug. 19, 2022.

(51) Int. Cl. A61B 5/00; A61M 16/00

(52) U.S. Cl. CPC A61B 5/4833; A61B 5/4815; A61B 5/746; A61B 5/7465; A61M 16/0051; A61M 16/024; A61M 2205/502; A61M 2205/84

(57) ABSTRACT

A method and system for providing community support for the primary user of a respiratory device is disclosed. Operational data from use of a respiratory therapy device by a primary user in accordance with complying with a respiratory treatment plan is collected. A secondary user, such as a friend or family member is linked to the primary user. Effectiveness data is determined from the collected operational data. The effectiveness data shows the effectiveness of the respiratory therapy device in relation to the respiratory treatment plan. The effectiveness data is sent to a user device operated by the secondary user. An interface is generated to allow the secondary user to view the effectiveness data.

### CROSS-REFERENCE TO RELATED APPLICATIONS

This application claims the benefit of, and priority to, U.S. Provisional Patent Application No. 63/371,973, filed Aug. 19, 2022, which is hereby incorporated by reference herein in its entirety.

### TECHNICAL FIELD

The present disclosure relates generally to systems and methods for respiratory therapy, and more particularly, to systems and methods for collaboration to assist in sleep therapy device use.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders are often treated using a respiratory therapy system (e.g., a continuous positive airway pressure (CPAP) system), which delivers pressurized air to aid in preventing the individual's airway from narrowing or collapsing during sleep. However, some users find such systems to be uncomfortable, difficult to use, expensive, aesthetically unappealing and/or fail to perceive the benefits associated with using the system. As a result, some users will elect not to use the respiratory therapy system or discontinue use of the respiratory therapy system absent a demonstration of the severity of their symptoms when respiratory therapy treatment is not used or encouragement or affirmation that the respiratory therapy system is improving their sleep quality and reducing the symptoms of these disorders. Many users feel isolated and/or stigmatized based on the PAP device usage. For example, a user may feel like their family doesn't understand, or isn't connected to, their therapy. This isolation results in diminished therapy usage. The present disclosure is directed to solving these and other problems.

### SUMMARY

According to some implementations of the present disclosure, a method includes collecting operational data from use of a respiratory therapy device by a primary user in accordance with complying with a respiratory treatment plan. A secondary user is linked to the primary user. Effectiveness data is determined from the collected operational data. The effectiveness data is sent to a user device operated by the secondary user. An interface is generated to allow the secondary user to view the effectiveness data.

According to some implementations of the present disclosure, a system includes a control system comprising one or more processors and a memory having stored thereon machine readable instructions. The control system is coupled to the memory and the above referenced methods are implemented when the machine executable instructions in the memory are executed by at least one of the one or more processors of the control system.

According to some implementations of the present disclosure, a computer program product comprises instructions which, when executed by a computer, cause the computer to carry out the above mentioned methods.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3A is a perspective view of a respiratory therapy device of the system of FIG. 1, according to some implementations of the present disclosure;

FIG. 3B is a perspective view of the respiratory therapy device of FIG. 3A illustrating an interior of a housing, according to some implementations of the present disclosure;

FIG. 4 is a block diagram of a system to allow secondary users to support a primary user of a respiratory therapy device, according to some implementations of the present disclosure;

FIG. 5A is a diagram of example interfaces generated by an application to assist in monitoring use of the respiratory therapy device for the primary user;

FIG. 5B is a diagram of example interfaces generated by an application for a secondary user to assist a primary user is using the respiratory therapy device; and

FIG. 6 is a process flow diagram for a method for communicating with secondary users, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

In this example, a system allows communication between a primary user of a respiratory therapy device and secondary users in an application for patient engagement such as the my Air application available from ResMed. The primary user is the respiratory therapy device user and the secondary user(s) can be a partner/spouse, family member, or friend linked to or associated with the primary user. Operational data from use of the respiratory therapy device by the primary user in accordance with complying with a respiratory treatment plan is collected. Effectiveness data relating to use of the respiratory therapy device in relation to the treatment plan is determined from the operational data. The secondary user is linked to the primary user's account such that the secondary user can have access to some or all of the primary user's sleep data, thus engaging the user's family. For example, effectiveness data may be sent to another application run by a device associated with the secondary user. Through the device, the secondary user can also provide insights, feedback, evaluation (e.g., log snoring, grumpometer, etc.), etc. By engaging family members and friends, the therapy device user can see the impact of their therapy device use on their families and friends. Additionally, the secondary users can help the primary user stay engaged. As one example, a combined sleep score can be provided (i.e., combined for the therapy device user and their bed partner). This can also be useful to keep track of family members (e.g., an elderly parent).

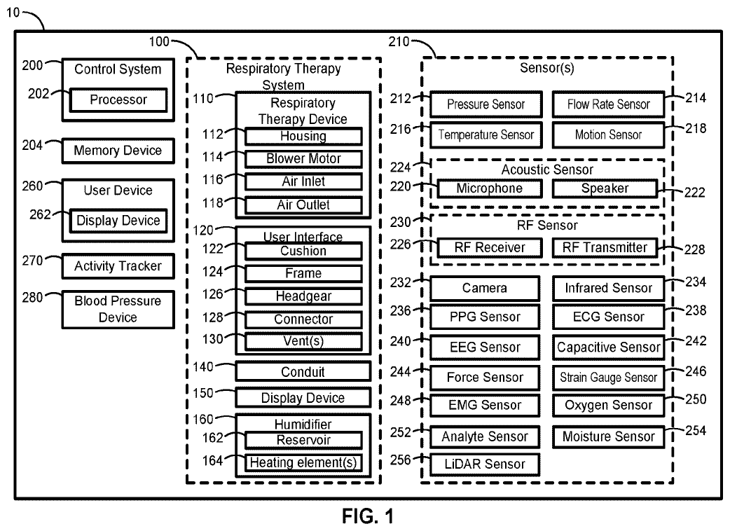

Referring to FIG. 1, a system 10, according to some implementations of the present disclosure, is illustrated. The system 10 includes a respiratory therapy system 100, a control system 200, one or more sensors 210, a user device 260, and an activity tracker 270.

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

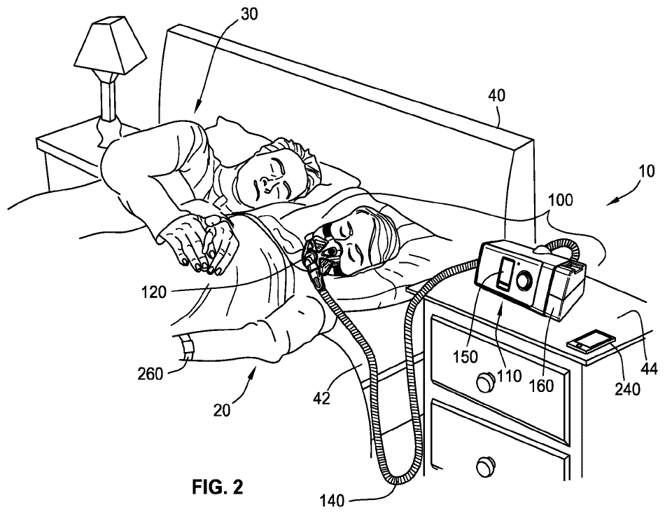

As shown in FIG. 2, the respiratory therapy system 100 can be used to treat user 20. In this example, the user 20 of the respiratory therapy system 100 and a bed partner 30 are located in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIG. 2, or more generally, on any surface or structure that is generally adjacent to the bed 40 and/or the user 20.

The respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂ O, between about 6 cmH₂O and about 10 cmH₂O, between about 7 cmH₂O and about 12 cmH₂ O, etc. The respiratory therapy device 110 can also deliver pressurized air at a predetermined flow rate between, for example, about −20 L/min and about 150 L/min, while maintaining a positive pressure (relative to the ambient pressure).

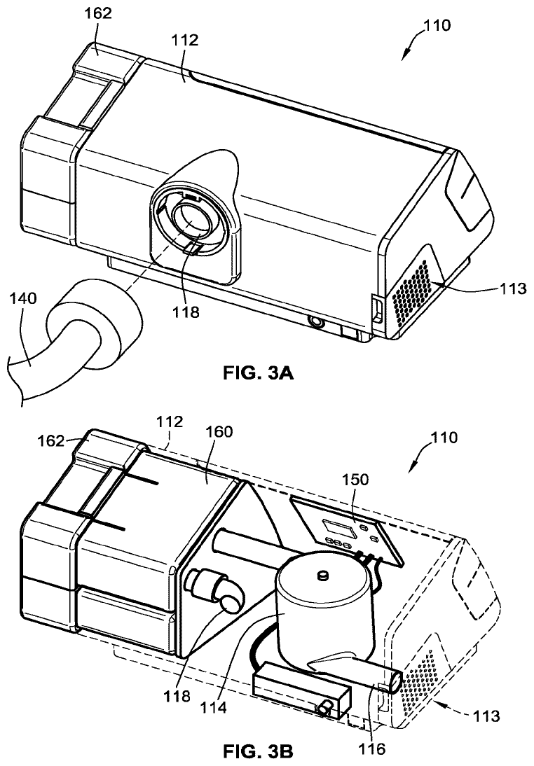

The respiratory therapy device 110 includes a housing 112, a blower motor 114, an air inlet 116, and an air outlet 118 (FIG. 1). Referring to FIGS. 3A and 3B, the blower motor 114 is at least partially disposed or integrated within the housing 112. The blower motor 114 draws air from outside the housing 112 (e.g., atmosphere) via the air inlet 116 and causes pressurized air to flow through the humidifier 160, and through the air outlet 118. In some implementations, the air inlet 116 and/or the air outlet 118 include a cover that is moveable between a closed position and an open position (e.g., to prevent or inhibit air from flowing through the air inlet 116 or the air outlet 118). As shown in FIGS. 3A and 3B, the housing 112 can include a vent 113 to allow air to pass through the housing 112 to the air inlet 116. As described below, the conduit 140 is coupled to the air outlet 118 of the respiratory therapy device 110.

Referring back to FIG. 1, the user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The vent 130 can be used for permitting the escape of carbon dioxide and other gases exhaled by the user 20. The user interface 120 generally can include any suitable number of vents (e.g., one, two, five, ten, etc.).

As shown in FIG. 2, in some implementations, the user interface 120 is a facial mask (e.g., a full face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

Referring back to FIG. 1, the conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

Referring to FIG. 3A, the conduit 140 includes a first end 142 that is coupled to the air outlet 118 of the respiratory therapy device 110. The first end 142 can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end 142 can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a my Air™ score, such as described in WO 2016/061629 and U.S. Patent Pub. No. 2017/0311879, which are hereby incorporated by reference herein in their entireties, the current date/time, personal information for the user 20, etc.). In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. For example, as shown in FIG. 3, air flow from the air inlet 116 through the blower motor 114, and then through the humidifier 160 before exiting the respiratory therapy device 110 via the air outlet 118.

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct).

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

Referring to back to FIG. 1, the one or more sensors 210 include a pressure sensor 212, a flow rate sensor 214, temperature sensor 216, a motion sensor 218, a microphone 220, a speaker 222, a radio-frequency (RF) receiver 226, a RF transmitter 228, a camera 232, an infrared sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a LiDAR sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the infrared sensor 234, the photoplethysmogram (PPG) sensor 236, the electrocardiogram (ECG) sensor 238, the electroencephalography (EEG) sensor 240, the capacitive sensor 242, the force sensor 244, the strain gauge sensor 246, the electromyography (EMG) sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 (FIG. 2) during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleet stage, an apnea-hypopnea index (AHI), pressure settings of the respiratory therapy device 110, or any combination thereof. The one or more events can include snoring, apneas, central apneas, obstructive apneas, mixed apneas, hypopneas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20 (FIG. 2), a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 alternatively or additionally generates one or more signals representing bodily movement of the user, from which may be obtained a signal representing a sleep state of the user; for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones

The speaker 222 outputs sound waves that are audible to a user of the system 10 (e.g., the user 20 of FIG. 2). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260.

The microphone 220 and the speaker 222 can be used as separate devices. In some implementations, the microphone 220 and the speaker 222 can be combined into an acoustic sensor 224 (e.g., a SONAR sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30 (FIG. 2). Based at least in part on the data from the microphone 220 and/or the speaker 222, the control system 200 can determine a location of the user 20 (FIG. 2) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g. a RADAR sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user (FIG. 2), to determine air flow of the mouth and/or nose of the user, to determine a time when the user enters the bed (FIG. 2), and to determine a time when the user exits the bed. In some implementations, the camera 232 includes a wide angle lens or a fish eye lens.

The infrared (IR) sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated in the user interface 120 and/or the associated headgear (e.g., straps, etc.).

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 174 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 174 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The Light Detection and Ranging (LiDAR) sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 100, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 132 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 210, or from other types of data.

The user device 260 (FIG. 1) includes a display device 262. The user device 260 can be, for example, a mobile device such as a smart phone, a tablet, a gaming console, a smart watch, a laptop, or the like. Alternatively, the user device 260 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 100 also includes an activity tracker 270. The activity tracker 270 is generally used to aid in generating physiological data associated with the user. The activity tracker 270 can include one or more of the sensors 210 described herein, such as, for example, the motion sensor 138 (e.g., one or more accelerometers and/or gyroscopes), the PPG sensor 154, and/or the ECG sensor 156. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIG. 2, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 100 also includes a blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIG. 2, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 maybe positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp chirp, triangle, sinewave), other schemes such as PSK, FSK etc., pulsed continuous wave, and/or spread in ultra wideband ranges (which may include spreading, PRN codes or impulse systems).

While the control system 200 and the memory device 204 are described and shown in FIG. 1 as being a separate and distinct component of the system 100, in some implementations, the control system 200 and/or the memory device 204 are integrated in the user device 260 and/or the respiratory therapy device 110. Alternatively, in some implementations, the control system 200 or a portion thereof (e.g., the processor 202) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user 20 has laid or sat down in the bed 40 (or another area or object on which they intend to sleep), and has turned on the respiratory therapy device 110 and donned the user interface 120. The sleep session can thus include time periods (i) when the user 20 is using the respiratory therapy system 100, but before the user 20 attempts to fall asleep (for example when the user 20 lays in the bed 40 reading a book); (ii) when the user 20 begins trying to fall asleep but is still awake; (iii) when the user 20 is in a light sleep (also referred to as stage 1 and stage 2 of non-rapid eye movement (NREM) sleep); (iv) when the user 20 is in a deep sleep (also referred to as slow-wave sleep, SWS, or stage 3 of NREM sleep); (v) when the user 20 is in rapid eye movement (REM) sleep; (vi) when the user 20 is periodically awake between light sleep, deep sleep, or REM sleep; or (vii) when the user 20 wakes up and does not fall back asleep.

The sleep session is generally defined as ending once the user 20 removes the user interface 120, turns off the respiratory therapy device 110, and gets out of bed 40. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user 20, ending when the respiratory therapy device 110 stops supplying the pressurized air to the airway of the user 20, and including some or all of the time points in between, when the user 20 is asleep or awake.

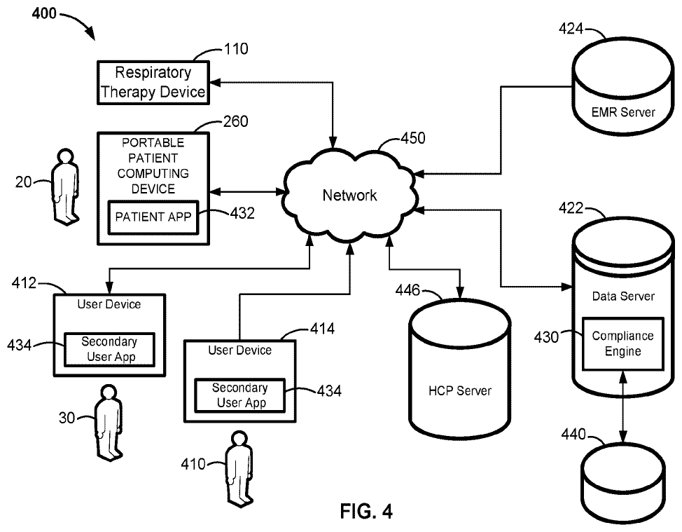

FIG. 4 shows a block diagram of an example support system 400 that allows community support for users of therapy devices. The support system 400 collects operational and usage data from respiratory therapy devices such as the respiratory therapy device 110 used by the primary user 20 in FIG. 2 in a general patient population. A health care system may include multiple patients such as the primary user 20. Each of the patients may be using a respiratory therapy device with functionality similar to the respiratory therapy device 110, additional sensors, such as a health monitoring device, and associated mobile computing devices. Related users, termed secondary users, that have a community relationship with the primary user 20 may also access the support system 400. Such secondary users may be linked to the primary user as will be explained below. In this example, the partner 30 shown in FIG. 2 is one secondary user. Another secondary user 410 may be a friend or family member of the primary user 20. Each of the secondary users 30 and 410 may operate a mobile computing user device such as mobile computing devices 412 and 414 respectively.

The underlying health care system includes a data server 422, an electronic medical records (EMR) server 424, and a health or home care provider (HCP) server 426. In this example, the data server 422 executes a compliance engine 430 and has access to different databases, such as the patient information database 440. The patient database 440 thus may include compliance data related to compliance by users, such as the user 20, with the respiratory therapy regimen using the respiratory therapy device 110.

These entities are all connected to, and configured to communicate with each other over the wide area network 450 such as the cloud or Internet. The connections to the wide area network 450 may be wired or wireless. The data server 422, EMR server 424, the HCP server 426, may all be implemented on distinct computing devices at separate locations, or any sub-combination of two or more of those entities may be co-implemented on the same computing device.

The servers 422, 424, and 426, are all a computer or network of computers. Although a simplified example is illustrated in FIG. 4, typically the application servers will be a server class system that uses powerful processors, large memory, and faster network components compared to a typical computing system used. The server typically has large secondary storage, for example, using a RAID (redundant array of independent disks) array and/or by establishing a relationship with an independent content delivery network (CDN) contracted to store, exchange and transmit data such as the asthma notifications contemplated above. Additionally, the computing system includes an operating system, for example, a UNIX operating system, LINUX operating system, or a WINDOWS operating system. The operating system manages the hardware and software resources of the application server and also provides various services, for example, process management, input/output of data, management of peripheral devices, and so on. The operating system provides various functions for managing files stored on a device, for example, creating a new file, moving or copying files, transferring files to a remote system, and so on.

The data server 422 includes a software architecture for supporting access and use of the compliance engine 430 by many different devices through the network 450, and thus at a high level can be generally characterized as a cloud-based system. Generally, the application server is designed to handle a wide variety of data. The application server includes logical routines that perform a variety of functions including checking the validity of the incoming data, parsing and formatting the data if necessary, passing the processed data to a database for storage, and confirming that the database has been updated.

In this example, either the respiratory therapy device 110 or the mobile device 260 associated with the primary user 20 is configured to intermediate between the primary user 20 and the remotely located entities of the health care system over the wide area network 450. In the implementation of FIG. 4, this intermediation is accomplished by a software application program 432 that runs on the mobile device 260 or on the respiratory therapy device 110. Such an application may be a dedicated application such as My Air™ or a web browser that interacts with a website provided by the health or home care provider. Related applications 434 may be executed by the mobile devices 410 and 412 to allow secondary users to obtain certain data relating to the primary user 20 and also provide support for the primary user 20.

Collected data received from the respiratory therapy device 110 or the mobile device 260 is stored and indexed in the database 440 by the data server 312 so as to be uniquely associated with the primary user 20 and therefore distinguishable from data collected from other devices in the system 400. In this regard, the system 400 may include many more respiratory therapy devices, sensors, mobile computing devices, and other components. The data server 422 may be configured to calculate summary data for each session from the data received from the respiratory therapy device 110. The data server 422 may also be configured to receive data from the mobile device 260, including data entered by the respective primary user 20, behavioral data about the primary user 20, or qualitative data.

The EMR server 424 contains electronic medical records (EMRs), both specific to the patients of the system and generic to a larger population of user with similar disorders to the primary user 20. An EMR, sometimes referred to as an electronic health record (EHR), typically contains a medical history of a patient, including previous conditions, treatments, co-morbidities, and current status. The EMR server 424 may be located, for example, at a hospital where any of the patients have previously received treatment. The EMR server 424 is configured to transmit EMR data to the data server 422, possibly in response to a query received from the data server 422.

In this example, the HCP server 426 is associated with the health/home care provider (which may be an individual health care professional or an organization) that is responsible for the patient's respiratory therapy. An HCP may also be referred to as a DME or HME (domestic/home medical equipment provider).

In some implementations, the data server 422 is configured to communicate with the HCP server 426 to trigger notifications or action recommendations to an agent of the HCP such as a nurse, or to support reporting of various kinds. Details of actions carried out are stored by the data server 422 as part of the engagement data.

For example, the compliance engine 430 provide compliance analysis based on the use of the respiratory therapy device 110 in accordance with compliance rules that specify the required usage over a compliance period, such as usage of the device for at least 4 hours per night on 70% of nights during any consecutive 30-day period within the first 90 days of therapy. The summary data post-processing may determine whether the most recent time period is a compliant session by comparing the usage time with the minimum duration from the compliance rule. The results of such post-processing are referred to as “compliance data.” Such compliance data may be used by a health care provider to tailor therapy that may include the respiratory therapy device 110 and other mechanisms. Other actors such as payors may use the compliance data to determine whether reimbursement may be made to a patient. Thus, in this example, the compliance engine 320 may be part of a diagnosis, compliance and therapy management application accessible a user device or a workstation associated with a healthcare provider. An example compliance and therapy management application may be the AirView™ application.

As may be appreciated, data in the data server 422, EMR server 424, and HCP server 426 is generally confidential data in relation to the patients in the system. Typically, patients must provide permission to send the confidential data to another party. Such permissions may be required to transfer data between servers 422, 424, and 426 if such servers are operated by different entities.

The example method and system 440 in FIG. 4 allows community support for primary user 20 of the respiratory therapy device 110 from one or more secondary users. One example of a secondary user offering community support may be a partner linked to the primary user, such as the bed partner 30 in FIG. 2. Other community support may be offered from any person that has a relationship with the user such as a family member or a friend such as the secondary user 410. Although only two secondary users are shown in FIG. 4 that are linked to the primary user 20, any number of secondary users may be linked to the primary user 20 to provide support. As will be explained, the example system 400 allows the primary user 20 communication with secondary users to avoid a primary user 20 from feeling isolated and alone when using the respiratory therapy device 110 for a respiratory ailment treatment program. The communication is based on sharing data such as effectiveness data determined from the operation of the respiratory therapy device 110 by the primary user 20 with one or more secondary users. Effectiveness data shows the effect of the respiratory therapy device 110 on the primary user 20 in accordance with complying with the respiratory ailment treatment program. The effectiveness data is derived from operational data from the respiratory therapy device 110 such as motor data, pressure sensor data, acoustic data, and the like. The effectiveness data may include data showing the times of operation of the respiratory therapy device 110 and corresponding comparison of compliance with the treatment plan. The effectiveness data may also include other analytics such as quality of sleep and duration of sleep for the primary user in relation to using the respiratory therapy device 110 in accordance with the treatment plan. For example, various sleep parameters derived from data collected by the sensors in FIG. 2 may be incorporated. Sleep session data may also be incorporated. A sleep score may also be determined as described herein to show the effectiveness of the respiratory therapy device 110.

The application 434 installed on the user devices 412 and 414 of the secondary users allows such secondary users to receive relevant effectiveness data relating to use of the respiratory therapy device 110 by the primary user 20. The secondary users may then employ the user devices 412 and 414 to offer support to the primary user 20 or be informed and otherwise contact the primary user to remotely offer support in real-time or otherwise. As explained above, each of the user devices 412 and 414 may execute the application 434 that allows any of the secondary users linked to the primary user 20 to receive effectiveness data related to the primary user 20. The data may be presented to show relevant compliance or non-compliance with the respiratory treatment plan. The application 434 also provides interfaces for the secondary users to provide support and encouragement for the primary user 20.

For example, the interface generated by the application 434 may display information to the secondary user including metrics of sleep quality, such as a sleep score, of the primary user 20, whether the respiratory therapy device 110 is being used, and the times the respiratory therapy device 110 is being used. Additional data may be derived from the application 432 on the user device 260 or the data server 422 such as predictions of compliance, occurrence of events such as apneas, number of events, number of hours used, AHI, leaks, etc., may be displayed for the secondary user. In addition, information in the form of pop-up displays or videos may be displayed showing the benefits of using the respiratory therapy device 110. The interface generated by the application 434 may further display data interpretation for the secondary user of the received information. For example, the application 434 may provide notifications of positive or negative trends in relation to effectiveness of the treatment or compliance with the respiratory treatment plan related to the received information from the respiratory therapy device 110.

The application 434 on the user devices 412 and 414 associated with the secondary users 30 and 410 may also include interfaces that allow a secondary user to send encouraging motivation to the primary user. The motivation may be in response to either a positive or a negative trend based on the data collected from the primary user and the respiratory therapy device 110. The application 434 may allow a secondary user, e.g., a partner, family member, or friend, to encourage support in the form of communication such as an email, a text message, emoji, gif, meme, or a video. The application 434 may generate an interface to access such applications on the user device and thus provide the options for communication through the application. Alternatively, the application 434 may provide links to open such communication applications on the user device.

Such messages may be composed by the secondary user or may be pre-selected from a menu generated by the application 434. Based on the data, the application may also remind the secondary user to send an encouraging message. The application 434 may also be set up to prompt the secondary user to view information such as a pop-up window or watch a video at a specific time to provide information to the secondary user to encourage the primary user. For example, the reminder and information relating to the importance of using the respiratory therapy device 110 may occur right before a bed time so the secondary user may communicate with the primary user to encourage the primary user to use the respiratory therapy device 110.

The application 432 may include an access control interface for the primary user to select secondary users to be linked to, and optionally, assign a tier of the secondary user. Thus, the primary user can control the number of secondary users through the application 432. The primary user may also control what data is shared with secondary users through their corresponding user devices and application 434 via a selection of one or more tiers for a secondary user. In this example, the level of the tier may be correlated with the types of data shared by the primary user. For example, a primary user may have a first tier of friends who are secondary users and a second higher tier of close family who are secondary users. The second higher tier allows the primary user to share more data with a secondary user such as the bed partner 30.

The primary user may also determine types of data to share with all secondary users. For example, the primary user may designate a secondary user to receive data on episodes, daily summaries, and use/non-use of the respiratory therapy only, while not sending data such as number of events per night, AHI, sleep times or the like. The secondary user may also select what types of data to receive from the primary user through the application 434. The primary user may also designate other actors in the system 400 for sharing information. For example, certain data may be shared with a workplace or a social organization. These actors may provide further support or encouragement to the primary user. For example, if an employee started respiratory therapy as a benefit from employment health benefits at a workplace, the employer or health care provider may be provided monitoring data, and based on results may provide additional incentives to the primary user. In another example, achieving a good sleep score may be desirable for a workplace. Such behavior may be incentivized to have good sleep prior to their shift so as to reduce the risk of traffic incidences or for higher productivity for professions such as drivers (trucks, rideshare, forklift), factory workers, or the like.

In this example, the primary user downloads the application 432 on the user device 260 in conjunction with use of the respiratory therapy device 110. The application 432 includes a community setup interface allowing the primary user to create accounts for secondary users to be linked to the primary user or send invitations to secondary users to join the community. The application 432 may also allow the primary user to select a tier for each secondary user and types of information that may be shared with each tier or individual secondary user. The application 432 allows sending invitations to a user device operated by a secondary user, such as the bed partner 30 or secondary user 410 in FIG. 4.

In this example, the data server 420 may manage an account for the primary user that may be related to other therapy plans managed by a health care organization. Data related to the primary user such as effectiveness data, demographic data, health records, and the like may be stored and associated with the account. Any of the linked secondary users may have access to the same account as the primary user, for example, if access has been granted by the primary user. The secondary users may have limited access to data such as effectiveness data for purposes of providing support from the primary user account. In this example, the primary user and all secondary users may share one account that allows access to common shared data. Alternatively, the primary user may select different levels of access to each member of the account and correspondingly what data is shared. For example, a family level member may access more data than a friend level member. Alternatively, a separate account may be provided for the secondary user that may allow access to certain data related to the primary user. The separate account may also store collection of health-related data and other data specific to the secondary user that may or may not be accessible to the primary user. A secondary user may be associated with multiple primary users. In such a case, the application 434 may allow the generation of multiple tiles on an interface to allow the secondary user to select which of the primary user data sets the secondary user wishes to view.

When the primary user selects a secondary user, a communication such as an email or text, may be sent to the potential secondary user. The communication includes a mechanism, such as a secure URL link, for the secondary user to download the application on their user device. Following the link allows the secondary user to download the application 434 and then set up their account or access the account of the primary user.

As explained above, secondary users may be assigned different tiers depending on the selection of the primary user. For example, the bed partner 30 may be assigned a higher tier than a friend such as the secondary user 410. Depending on the tier of the secondary user, additional communication and data features may be available that may not be available to a secondary user that is assigned a lower tier. For example, if the secondary user is a partner of the primary user, such as the bed partner 30, the application may enable additional data from the secondary user to be displayed to the primary user. Such information may include data relating to the impact of their sleep quality on the bed partner 30. Such information may not be available from a lower tier secondary user. This information may include analysis of the beneficial effects of adhering to the therapy plan for a secondary user of the higher tier. For example, the information may emphasize that the therapy based on use of the respiratory therapy device 110 prevented the bed partner 30 from stopping breathing X times last night or may have roused the bed partner 30. The information may also indicate that the lack of use of the respiratory therapy device 110 may have resulted in snoring of the primary user leading to the bed partner 30 waking up one or more times during the night. The sleep score of the bed partner 30 could also be displayed to the primary user 20 via the application 432, showing a high sleep score because of compliance or a low sleep score due to lack of compliance.

The application 434 for the secondary user may also allow the secondary user such as the bed partner 30 to log a reaction to their own sleep through either objective or subjective data. Such data may be correlated with a sleep score from the primary user and presented to the primary user via the application 432.

The secondary user may input subjective data through interfaces generated by the application 434. For example, the bed partner 30 may score the primary user 20 in relation to the sleep quality of the primary user 20 observed by the bed partner 30. The application may also allow the bed partner 30 to select levels of the quality of sleep or offer free form responses as to the quality of sleep of the primary user or themselves. Automatic objective inputs from a wearable such as fitness tracker or a smart watch worn by the secondary user 30 similar to those described in relation to the activity tracker 270 in FIG. 2 could automatically report sleep related data for the secondary user to the application 434 on the user device 412.

A comparison of the sleep data of the bed partner 30 during use of the respiratory therapy device 110 with sleep data when the device 110 is not in user may be displayed by the application 432 to the primary user to motivate better adherence. Such data from a secondary user such as the bed partner 30 may provide insight into the partner's sleep. The data may be correlated with collected operational data from the respiratory therapy device 110, to show that when a primary user disrupted use of the therapy device 110, such as taking the mask off, the bed partner 30 was woken up. Another example is showing trends of sleep related data from the secondary user over several days or weeks. For example, the primary user may be shown that a trend of better health occurs for the secondary user through charting sleep score or another health measure over a period of time. If physiological data related to activity is collected from a wearable such as step tracker, data related to increased activity (e.g., more steps taken or more workouts completed) as a result of better sleep for the secondary user may be displayed to the primary user to motivate the primary user. Another example is obtaining subjective data such as in the form of a survey from the secondary user. Such a survey would include questions for the secondary user to indicate that they have had good or bad sleep. The results could be correlated with the primary user's use of the respiratory therapy device 110.

Other methods may be used to determine sleep quality of the secondary user for purposes of comparison for the primary user. For example, the application 434 may allow facial image capture and analysis. The facial image of the secondary user 30 may be captured through an on-board camera on the mobile device 412 after a night of sleep. Facial features from a captured image may show level of sleep such as lack of bloodshot eyes, no bags under eyes, and other facial features. Such data may also be shown over a period of time to show trends of better sleep for the secondary user to motivate the primary user.

Another set of data that may provide support for the primary user may be a combined sleep score that may be shared between the primary user and the bed partner 30. In this example, the sleep score of the bed partner 30 may be derived from a wearable device with a sleep score application. The sleep score data of the bed partner 30 may be associated with the primary user to determine a combined sleep score.

In this example, both the primary user 20 and the secondary user 30 may view an interface on their respective mobile devices, generated by the respective applications 432 and 434 that shows the combined sleep score. Other data such as each person's contribution to the score may be shown as additional motivation for the primary user. The data may also be correlated with other health factors such as fitness. For example, if the data is correlated with user fitness, notifications to the primary/secondary user may be sent when one is sleeping better and has improved fitness or increased energy levels.

The sleep data of the secondary user may also be used to diagnosis sleep health of the secondary user. For example, sleep data of a bed partner such as the bed partner 30 may suggest that the partner may need respiratory therapy (e.g., a plan to use a respiratory therapy device) or may indicate a risk of a respiratory disease. Based on sleep data, alerts may be issued to the secondary user by the application 434 to prompt secondary users to get a sleep test or to complete a sleep survey. Thus, the data of the primary user may be used as a motivation to the secondary user as the effectiveness data related to the primary user may show how much better the primary user is doing because of therapy based on the respiratory therapy device.

Other features from the application 432 may be shared with the application 434 on the user device of the secondary user. For example, the application 432 may display a schedule or calendar that shows the primary user therapy reminders during the day or days of the therapy period. This calendar and corresponding data may be displayed by the application 434 on the user device of the secondary user. Sharing such reminder information allows for coordinated support from the secondary user with the primary user. For example, such scheduling information shared with secondary users keeps the secondary users and the primary user informed of the schedule of respiratory treatment. If the primary user has an appointment with a health care professional, therapist, or other actor, secondary users can remind the primary user. The calendar may also be used in conjunction with goals set for the primary user. The secondary user may be informed of goals and achievements through communications received by the application 434.

Another feature of the application 432 may be to offer helpful information or tips for the primary user 20 in relation to using the respiratory therapy device 110. Such tips may also be displayed for the secondary user simultaneously via the application 434. Such tips and information may allow discussion and encouragement between the secondary users and the primary user.

The application 432 may also import goals from the compliance plan and provide alerts when a goal is achieved by the primary user 20. The alerts may also be received by application 434 on the user device operated by a secondary user. The alerts allow secondary users to comment on the primary user 20 hitting milestones and achievements in relation to a treatment plan. Other alerts may be sent to the mobile device for the secondary user. For example, an alert may be sent when settings of the respiratory therapy device 110 are changed. There may be a threshold for the alert, such as a setting changed over 10%. An alert may also be sent when a mask type connected to respiratory therapy device 110 is changed. An alert may also be sent when a component of respiratory therapy device 110 requires replacement. Such alerts may provide a back up, through the secondary user, to ensure proper operation and use of the respiratory therapy device 110 by the primary user.

The application 434 may allow other forms of remote monitoring of the primary user 20 depending on the level of access allowed by the primary user. The access may be granted based on the tier of the secondary user, or the primary user may select different types of data that may be shared. For example, sensed data from a fitness tracker or other sensors may be sent to the application 434 to allow a secondary user to know when the primary user is going to sleep or waking up. The application 434 may also analyze the data to infer other information if habits or behavior of the primary user change.

The application 432 on the user device, the respiratory therapy device 110, or the compliance engine 430 may also provide alerts to secondary users in relation to the primary user 20. Such alerts may be triggered when an anomaly in the data related to the primary user is detected. For example, such anomalies may include excessive times the primary user wakes up, taking a long time to fall asleep, went to bed really late, and the like. Typically, the primary user is asleep when the anomaly happens, and thus informing the secondary user may allow for monitoring and alerting a health care professional about the anomaly.

The application 432 may allow an alert to be sent to the secondary users to either check on the primary user and/or contact a health care professional. Other alerts may be triggered for long term trends. For example, the secondary user may be alerted when the primary user stops using therapy regularly over a period of time. Another example may be sleep times over a period of time that show that the primary user is going to bed later and later or having insufficient quality sleep.

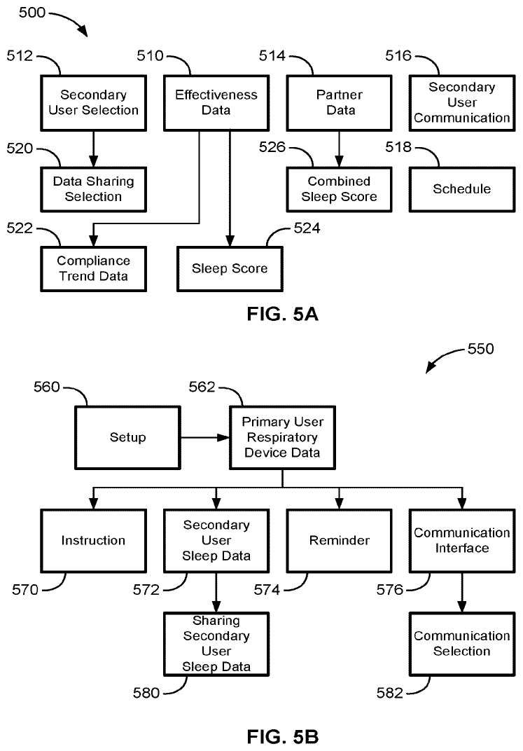

FIG. 5A is a screen interface diagram 500 for the application 432 that may be operated by the primary user 20 in FIG. 4. The interface diagram 500 shows some example interfaces that may be generated by the application 432 for purposes of community support. The application 432 may generate one of a set of initial interfaces including an effectiveness data interface 510, a secondary user selection interface 512, a partner data interface 514, a secondary user communication interface 516, and a schedule interface 518. The effectiveness data interface 510 that displays effectiveness data relating to the use of the respiratory therapy device 110 by the primary user 20. The secondary user selection interface 512 allows the primary user to select different individuals to serve as secondary users for a support community. The secondary user interface 512 may display contacts available from other applications on the user device and/or allow the primary user to enter information for secondary users. Secondary user data entered through the secondary user interface 512 allows communication to be established with potential secondary users and the application 434 to be downloaded to corresponding user devices. A data selection interface 520 may be accessed through secondary user interface 512 that allows the primary user to select the types of data shared with the secondary user.

The partner data interface 514 displays data from the secondary user that may be helpful to motivate behavior such as sleep score, health data, or sleep times. The secondary user communication interface 516 is activated when motivating communications are received from the application 434 executed by the user device of a secondary user. The schedule interface 518 may display a calendar of the treatment plan and use times of the respiratory therapy device. The schedule interface 518 may be synched with a similar interface generated by the application 434 for the secondary user.

FIG. 5B is a screen interface diagram 550 for the application 434 that may be operated by a secondary user. A set of initial interfaces may be generated by the application 434. The initial interfaces include a set up interface 560 that allows a secondary user to enter account information to link themselves to the primary user. Once the application 434 is setup, a data interface 562 is displayed. The data interface 562 shows effectiveness data from the application 432 relating to the primary user and the use of the respiratory therapy device 110. Other data may also be displayed on the interface in accordance with the data a primary user selects that will be shared with a secondary user.

In response to effectiveness data, a secondary user may access a series of interfaces to assist in motivating the primary user. The series of interfaces includes an instruction interface 570, a secondary user sleep data interface 572, a reminder interface 574, and a communication interface 576. The instruction interface 570 may allow the secondary user to select available media for additional information for the benefits of the respiratory therapy device 110 for the primary user. The secondary user sleep data interface 572 shows data relating to sleep for the secondary user. This data may be determined via subjective input through answering a survey displayed by the application 434 and/or by incorporating sensor data from a wearable device or other sensors. A sharing data interface 580 may be accessed through the sleep data interface 572 that allows the secondary user to share their sleep data with the primary user. The reminder interface 574 may display a common schedule or calendar that shows the dates and times the treatment plan requires use of the respiratory therapy device 110. The reminder interface 574 may allow the secondary user to set a reminder that will be generated by the application 432 for the primary user. The communication interface 576 allows the secondary user to communicate with the primary user. Thus, the communication interface 576 may access other communication applications such as phone, email, text, or video on the user device. A communication selection interface 582 may be accessed through the communication interface 576 that allows the secondary user to select specific pre-selected content for the communication.

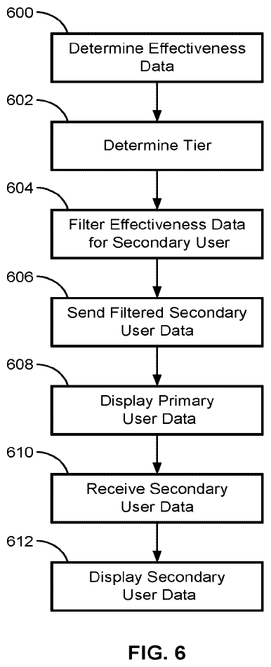

FIG. 6 shows a flow diagram for the community support routine performed by the system 400. The flow diagram in FIG. 6 is representative of an example routine implementable by machine-readable instructions for the system 400 in FIG. 6. In this example, the machine-readable instructions comprise an algorithm for execution by (a) a processor; (b) a controller; and/or (c) one or more other suitable processing device(s). The algorithm may be embodied in software stored on tangible media such as flash memory, CD-ROM, floppy disk, hard drive, solid-state drive, digital video (versatile) disk (DVD), or other memory devices. However, persons of ordinary skill in the art will readily appreciate that the entire algorithm and/or parts thereof can alternatively be executed by a device other than a processor and/or embodied in firmware or dedicated hardware in a well-known manner (e.g., it may be implemented by an application-specific integrated circuit (ASIC), a programmable logic device (PLD), a field-programmable logic device (FPLD), a field-programmable gate array (FPGA), discrete logic, etc.). For example, any or all of the components of the interfaces can be implemented by software, hardware, and/or firmware. Also, some or all of the machine-readable instructions represented by the flowcharts may be implemented manually. Further, although the example algorithm is described with reference to the flowchart illustrated in FIG. 6, persons of ordinary skill in the art will readily appreciate that many other methods of implementing the example machine-readable instructions may alternatively be used. For example, the order of execution of the blocks may be changed, and/or some of the blocks described may be changed, eliminated, or combined.

The routine will determine effectiveness data associated with the primary user based on collected operational data from the respiratory therapy device 110 and other relevant data sources (600). The routine then determines the tier of a secondary user (602). The routine then filters the effectiveness data according to the determined tier of the secondary user (604). The filtered data is then sent to the user device operated by the secondary user (606). The routine will then display the filtered effectiveness data to the secondary user (608). The routine will receive any relevant data, such as sleep score, from the user device of the secondary user (610). The routine may then display the secondary user data to the primary user (612).

One or more elements or aspects or steps, or any portion(s) thereof, from one or more of any of claims 1 to 28 below can be combined with one or more elements or aspects or steps, or any portion(s) thereof, from one or more of any of the other claims 1 to 28 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
collecting operational data from use of a respiratory therapy device by a primary user in accordance with complying with a respiratory treatment plan;
linking a secondary user to the primary user;
determining effectiveness data from the collected operational data;
sending the effectiveness data to a user device operated by a secondary user; and
generating an interface to allow the secondary user to view the effectiveness data.

2. The method of claim 1, wherein the secondary user is a family member or a friend or an individual in proximity to the primary user when using the respiratory therapy device.

3. (canceled)

4. The method of claim 1, wherein the effectiveness data includes a sleep score for the primary user.

5. The method of claim 4, further comprising:
determining a sleep score for the secondary user; and
determining a combined sleep score based on the sleep score of the primary user and the sleep score of the secondary user.

6. The method of claim 5, wherein the sleep score of the secondary user is determined based on one of subjective data from the secondary user or wearable device data.

7. The method of claim 5, further comprising displaying the determined combined sleep score on a user device operated by the primary user.

8. The method of claim 2, further comprising measuring a health parameter of the secondary user; and displaying the health parameter of the secondary user on a user device operated by the primary user.

9. (canceled)

10. The method of claim 2, wherein sleep data is collected from the secondary user and displayed on a user device operated by the primary user.

11. The method of claim 2, wherein the user device operated by the secondary user includes an input interface for input of subjective information from the secondary user in relation to quality of sleep, wherein the method further comprises displaying the subjective information on a user device operated by the primary user.

12. The method of claim 1, further comprising classifying at least two tiers of secondary users, wherein each tier of secondary users obtains different levels of the effectiveness data.

13. The method of claim 1, wherein the effectiveness data includes whether the primary user is using the respiratory therapy device in accordance with a compliance plan.

14. The method of claim 1, wherein the user device includes an interface for the secondary user to communicate with the primary user to encourage the primary user to use the respiratory therapy device.

15-16. (canceled)

17. The method of claim 1, wherein the effectiveness data includes use of the respiratory therapy device over a series of uses by the primary user, and wherein positive or negative trends in relation to the compliance plan are determined.

18. The method of claim 1, further comprising displaying a schedule of the primary user using the respiratory therapy device on the user device.

19. The method of claim 18, further comprising sending an alert to the user device based on a schedule of the primary user using the respiratory therapy device, when any settings of the respiratory therapy device are changed, or when a mask type connected to respiratory therapy device is changed.

20-21. (canceled)

22. The method of claim 1, further comprising sending an alert to the user device when a component of respiratory therapy device requires replacement.

23. The method of claim 1, further comprising:
determining an anomaly in sleep from the collected data; and
alerting the user device of the secondary user.

24. The method of claim 1, wherein the linking of the secondary user includes allowing the primary user to select the secondary user and contacting the user device to install an application.

25. A system comprising:
a control system comprising one or more processors; and
a memory having stored thereon machine readable instructions;
wherein the control system is coupled to the memory, and when the machine executable instructions in the memory are executed by at least one of the one or more processors of the control system, the one or more processors are operable to:
collect operational data from use of a respiratory therapy device by a primary user in accordance with complying with a respiratory treatment plan;
link a secondary user to the primary user;
determine effectiveness data from the collected operational data;
send the effectiveness data to a user device operated by a secondary user; and
generate an interface to allow the secondary user to view the effectiveness data.

26. (canceled)

27. A non-transitory computer readable medium computer program product comprising instructions which, when executed by a computer, cause the computer to carry out:
collecting operational data from use of a respiratory therapy device by a primary user in accordance with complying with a respiratory treatment plan;
linking a secondary user to the primary user;
determining effectiveness data from the collected operational data;
sending the effectiveness data to a user device operated by a secondary user; and
generating an interface to allow the secondary user to view the effectiveness data.

28. (canceled)
